(19) United States
(12) Patent Application Publication
(10) Pub. No.: US 2021/0128439 A1
(43) Pub. Date: May 6, 2021

(54) Edible Film

(71) Applicant: MORINAGA MILK INDUSTRY CO., LTD., Tokyo (JP)

(72) Inventors: Tomohito Ozawa, Kanagawa (JP); Manabu Nakano, Kanagawa (JP); Nobuo Seki, Kanagawa (JP)

(73) Assignee: MORINAGA MILK INDUSTRY CO., LTD., Tokyo (JP)

(21) Appl. No.: 16/635,811

(22) PCT Filed: Aug. 2, 2018
(86) PCT No.: PCT/JP2018/029027

§ 371 Date: Jan. 31, 2020

(30) Foreign Application Priority Data
Aug. 3, 2017 (JP) 2017-150751

(51) Int. Cl. A61K 8/66; A61K 8/73; A61K 8/02; A61K 8/60; A61K 8/34; A61K 8/64; A61K 8/365; A61Q 11/00; A23L 29/00; A23L 29/30; A23L 33/19; A23P 20/20; A23G 3/36; A23G 3/42; A23G 3/44; A23G 3/54

(52) U.S. Cl. CPC A61K 8/66; A23V 2002/00; A61K 8/0216; A61K 8/60; A61K 8/34; A61K 8/0233; A61K 8/64; A61K 8/345; A61K 8/365; A61K 8/602; A61K 8/731; A61Q 11/00; A23L 29/06; A23L 29/37; A23L 33/19; A23P 20/20; A23G 3/366; A23G 3/42; A23G 3/44; A23G 3/54; A61K 8/732

(57) ABSTRACT

An object of the present invention is to provide an edible film that can be kept for a long time in the oral cavity and ingested readily. Additionally, another object of the present invention is to provide an edible film that does not degrade the activity of the enzyme contained in the edible film for a long period of time. The objects are achieved by an edible film comprising lactoperoxidase, glucose oxidase, a glucose source, and a film former.

### TECHNICAL FIELD

The present invention relates to an edible film comprising lactoperoxidase, glucose oxidase, a glucose source, and a film former.

### BACKGROUND ART

It has been known that compositions comprising lactoperoxidase, glucose oxidase, and a glucose source are traditionally used as oral disinfectants, and are useful in the prevention and treatment of periodontal disease, halitosis, and aspiration pneumonia. Of these, lactoperoxidase is known to catalyze the generation of hypothiocyanic acid in the presence of hydrogen peroxide and thiocyanate, exhibiting strong antimicrobial activity. The system that exhibits antibacterial activity is referred to as the lactoperoxidase system. On the other hand, this lactoperoxidase system is known to exhibit strong bactericidal activity even in the absence of thiocyanate (Patent document 1).

Additionally, it has been disclosed that compositions containing lactoperoxidase, glucose oxidase, and a glucose source can be used as a sulfur-containing amino acid lyase inhibitor (Patent document 2), and can also be used as antiviral agents (Patent document 3).

Furthermore, it has been disclosed that a composition in which lactoferrin is added to lactoperoxidase, glucose oxidase, and a glucose source was used as an upper respiratory tract protectant, and that it was useful in removing foreign objects from the upper respiratory tract, preventing foreign objects from invading into the upper respiratory tract, moisturizing the upper respiratory tract, suppressing mucosal drying of the upper respiratory tract, reducing mucosal irritation in the upper respiratory tract, preventing and/or reducing cold symptoms (Patent document 4). In addition, it has been disclosed that the effect of long-term ingestion of tablet confectionaries containing lactoferrin, lactoperoxidase, glucose oxidase, and a glucose source as the active ingredients is to improve the state of bacterial flora in the oral cavity (Non-patent document 1).

In addition, it has been disclosed that the long-term ingestion of tablet confectionaries containing lactoferrin, lactoperoxidase, glucose oxidase, and a glucose source as active ingredients achieved “the effects of improving the state of saliva (such as the amount of saliva, smooth feeling, etc.)” and “the effects of improving the whole body (chill, cold, and hay fever, etc.)” as a somatic experience (Non-patent document 2).

When using compositions comprising lactoferrin, lactoperoxidase, glucose oxidase, and a glucose source, it is desirable that the compositions remain in the oral cavity for a long time, be in a product form that is convenient to carry, have good preservation stability, and be able to be readily ingested.

On the other hand, various edible films are used in the food, pharmaceutical fields, etc. Many of them are edible films made of film-like substrates, etc., which dissolve quickly in the oral cavity and do not remain for a long time in the oral cavity (for example, Patent document 5).

### CITATION LIST

### Patent Documents

Patent document 1: Japanese patent No. 4203120

Patent document 2: Japanese Laid-open (kokai) Patent Application Publication No. 2015-149904

Patent document 3: Japanese patent No. 4355592

Patent document 4: WO 2017/033616A1

Patent document 5: Japanese Laid-open (kokai) Patent Application Publication No. 2016-047802

### Non-Patent Documents

Non-Patent document 1: Manabu Nakano et al., Milk Science, 2016, Vol. 65, No. 3, Pages 227-234

Non-patent document 2: Eiju Shimizu et al., The Journal of Japan Dental Society of Oriental Medicine, 2011, Vol. 30, 1 and 2, Pages 1-7

### SUMMARY OF INVENTION

### Technical Problem

The present invention was made in light of the aforementioned circumstances. An object of the present invention is to provide an edible film that can be kept for a long time in the oral cavity and ingested readily. Additionally, another object of the present invention is to provide an edible film that does not degrade the activity of the enzyme contained in the edible film for a long period of time.

### Solution to Problem

The present inventors discovered that, as a result of an intense study to solve the problems, the objects can be solved by the inclusion of a film former that remains for a long time in the oral cavity as a part of an edible film.

That is, the present invention relates to an edible film comprising lactoperoxidase, glucose oxidase, a glucose source, and a film former.

Preferably, the edible film is monolayered.

Additionally, the edible film is preferably multilayered and does not include the glucose source and glucose oxidase in the same layer.

Additionally, the edible film preferably has a lactoperoxidase content of 0.02 to 20 wt %, a glucose oxidase content of 0.02 to 20 wt %, and a glucose source content of 0.02 to 40 wt %.

Additionally, the edible film preferably further comprises lactoferrin, and the edible film preferably has a lactoferrin content of 0.02 to 40 wt %.

Furthermore, the edible film preferably consists of one or more selected from the group consisting of pullulan, hydroxypropyl cellulose (HPC), hydroxypropyl methylcellulose (HPMC), processed starch, and porphyran.

The present invention also relates to a method for manufacturing an edible film, the method comprising: a step of forming into a film a mixture comprising lactoperoxidase, glucose oxidase, a glucose source, and a film former.

The present invention also relates to a method for manufacturing an edible film, the method comprising: a step of forming into a film either a first mixture or a second mixture, the first mixture comprising glucose oxidase without any glucose source, the second mixture comprising a glucose source without glucose oxidase; and a step of laminating, onto the mixture formed into the film, the other mixture, wherein either or both of the first mixture and the second mixture include lactoperoxidase, and either or both of the first mixture and the second mixture include a film former.

The present invention also relates to a method for manufacturing an edible film, the method comprising: a step of forming into a film a first mixture comprising glucose oxidase without any glucose source; a step of forming into a film a second mixture comprising a glucose source without glucose oxidase; and a step of laminating the first mixture formed into the film and the second mixture formed into the film, wherein the order in which the step of forming the first mixture into the film and the step of forming the second mixture into the film are performed is not limited, either or both of the first mixture and the second mixture include lactoperoxidase, and either or both of the first mixture and the second mixture include a film former.

### Advantageous Effects of Invention

According to the present invention, an edible film that is convenient to carry, that can be kept for a long time in the oral cavity for an extended period of time, and that can be readily ingested is provided. Additionally, in a preferred embodiment of the present invention, it is possible to provide an edible film comprising lactoperoxidase, glucose oxidase, and a glucose source, in which a decrease in activity of both enzymes is suppressed. The edible film is suitable for use as food and formulation.

### DESCRIPTION OF EMBODIMENTS

Next, a preferred embodiment of the present invention will be described in detail. However, the present invention is not limited to the following preferable embodiment and can be freely modified within the scope of the present invention. The percentages mentioned in this description are used on mass-basis unless especially indicated.

<Lactoferrin>

The edible film of the present invention preferably comprises lactoferrin. Lactoferrin is a type of milk protein. Lactoferrin used in the present invention can be obtained from mammalian milk, etc. In addition, commercially available products manufactured by a regular method can be used.

<Lactoperoxidase>

Lactoperoxidase is a type of milk protein and is a redox enzyme. Lactoperoxidase used in the present invention can be obtained, for example, from mammalian milk, etc. In addition, commercially available products manufactured by a regular method can be used.

<Glucose Oxidase>

Glucose oxidase is an enzyme that oxidizes glucose to produce gluconolactone, and glucose oxidase used in the present invention includes both enzymes produced, for example, by microorganisms, and those commercially available.

<Glucose Source>

The glucose source is, for example, glucose, or a combination of saccharides that contain glucose units and can produce glucose, and enzymes that decompose the saccharides to produce glucose. Such saccharides include oligosaccharides and polysaccharides. The enzymes include amylase and maltase. Glucose is preferred among these, and commercially available glucose can be used.

<Film Former>

The main component of the edible film of the present invention is a film former (film-forming agent). The film former of the present invention may form an edible film and dissolve in saliva when placed in an oral cavity. The material of a film former may be pullulan, guar gum, xanthan gum, alginates such as sodium alginate, or the like, gelatin, starches such as processed starch; maltodextrin, wheat gluten, carrageenan, locust bean gum, hydroxypropyl cellulose (HPC), hydroxypropyl methylcellulose (HPMC), pectin, and porphyran. Among these, pullulan, hydroxypropyl cellulose (HPC), hydroxypropyl methylcellulose (HPMC), processed starch, and porphyran are preferable. A film former used in the present invention may consist of one material or it may consist of a plurality of materials.

<Edible Film>

The film thickness of the edible film of the present invention is from 0.1 μm to 1000 μm.

The edible film of the present invention may comprise lactoperoxidase, glucose oxidase, a glucose source, and a film former described above, and in a preferred embodiment, the film may further contain lactoferrin described above.

A lactoperoxidase content in the edible film of the present invention is preferably 0.02 to 20 wt %. In addition, a glucose oxidase content is preferably 0.02 to 20 wt %. A glucose source content is preferably 0.02 to 40 wt %.

Additionally, when the edible film contains lactoferrin, a lactoferrin content is preferably 0.02 to 40 wt %.

The edible film of the present invention may be a monolayer comprising all components of lactoperoxidase, glucose oxidase, a glucose source, and a film former, or may be a multilayer in which components are divided.

In case of a multilayer, as long as the glucose source and glucose oxidase are not included in the same layer, and the effect of the edible film of the present invention is demonstrated, there is no limitation, but it is preferably in two layers. Lactoperoxidase and a film former, as well as lactoferrin in a preferred embodiment, may each be included in one or more layers and may be included in all layers. In the case of multiple layers, by the design in which a glucose source and glucose oxidase are not included in the same layer, decrease in the enzyme activity of glucose oxidase and lactoperoxidase can be suppressed for a longer period of time, and the physiological effects exerted on the person who has ingested the edible film will last longer.

Here, if the layer comprising the same ingredient is laminated continuously, it is defined as a monolayer rather than a multilayer.

The edible film of the present invention can take the form of a capsule formulation, for example, instead of the form of film. In the present invention, the edible film of the present invention is also referred to as an edible film even if the film is in the form of a capsule formulation. In the case of a capsule formulation, it may be devised so that a mixture containing glucose oxidase without any glucose source is made into an encapsulating component, while a mixture containing a glucose source without glucose oxidase is made into a component to be encapsulated, and vice versa.

Additionally, the edible film of the present invention can be an intraoral film-like formulation, or an intraoral film-like food or drink.

The edible film of the present invention can be utilized as an edible film for one or more applications selected from the group consisting of intraoral sterilization, sulfur-containing amino acid lyase inhibitor, anti-virus, protection of upper respiratory tract, removal of foreign objects from the upper respiratory tract, prevention of foreign objects from invading into the upper respiratory tract, moisturizing of the upper respiratory tract, suppression of mucosal drying of the upper respiratory tract, reduction of mucosal irritation in the upper respiratory tract, improvement of oral flora and improvement of saliva (for example, the amount of saliva, viscosity and smooth sensation of the saliva). Additionally, the edible film of the present invention can be utilized as an edible film for prevention or treatment of one or more diseases or symptoms selected from the group consisting of periodontal disease, halitosis, aspiration pneumonia, cold symptoms, chill, cold, and hay fever. Treatment includes improvement and alleviation.

<Method for Manufacturing an Edible Film>

A first embodiment of a method for manufacturing an edible film of the present invention is a method for manufacturing an edible film, the method comprising: a step of forming into a film a mixture comprising lactoperoxidase, glucose oxidase, a glucose source, and a film former.

A second embodiment of a method for manufacturing an edible film of the present invention is a method for manufacturing an edible film, the method comprising: a step of forming into a film either a first mixture or a second mixture, the first mixture comprising glucose oxidase without any glucose source, the second mixture comprising a glucose source without glucose oxidase; and a step of laminating, onto the mixture formed into the film, the other mixture, wherein either or both of the first mixture and the second mixture include lactoperoxidase, and either or both of the first mixture and the second mixture include a film former.

The edible film manufactured in the second embodiment may have any number of layers as long as the film is multilayered, and preferably has two layers.

In the case of three or more layers, the third and subsequent layers, including the third layer, can be handled in the same manner as the first mixture and the second mixture if the glucose source and glucose oxidase are not in the same layer, and the order in which the layers are laminated is not restricted. Additionally, any layer of the third or subsequent layer may or may not comprise lactoperoxidase, and may or may not include a film former.

The third embodiment of the method for manufacturing an edible film is a method for manufacturing an edible film, the method comprising: a step of forming into a film a first mixture comprising glucose oxidase without any glucose source; a step of forming into a film a second mixture comprising a glucose source without glucose oxidase; and a step of laminating the first mixture formed into the film and the second mixture formed into the film, wherein the order in which the step of forming the first mixture into the film and the step of forming the second mixture into the film are preformed is not limited, either or both of the first mixture and the second mixture include lactoperoxidase, and either or both of the first mixture and the second mixture include a film former.

Methods of laminating include, for example, a method for laminating a first mixture formed into films with a second mixture formed into films.

The edible film manufactured in the third embodiment may have any number of layers as long as the film is multilayered, and preferably has two layers.

In the case of three or more layers, the third and subsequent layers, including the third layer, can be handled in the same manner as the first mixture and the second mixture, if a glucose source and glucose oxidase are not included in the same layer. Additionally, any layer of the third or subsequent layer may or may not comprise lactoperoxidase, or may or may not include a film former.

Furthermore, the edible film of the present invention is not limited to a planar shape; it can be in a form of a three-dimensional shape, such as a capsule formulation, as long as it is a form that allows the product to be kept in the oral cavity for a long time and be ingested readily. In the case of a capsule formulation, etc., a mixture containing lactoperoxidase, glucose oxidase, a glucose source, and a film former may be molded into a capsule formulation by a normal method. Specific examples include embodiments of encapsulating a mixture containing glucose oxidase without any glucose source in a mixture containing a glucose source without glucose oxidase, or vice versa.

The edible film of the present invention may include components other than lactoperoxidase, glucose oxidase, a glucose source, and a film former. A component that can be included in an edible film of the present invention is preferably lactoferrin. Examples of other ingredients include caffeine, xylitol, guava leaf extract, Japanese pepper extract, clove oil, iron chlorophyllin sodium, mint oil, Flavangenol, peppermint, menthol, eucalyptus oil, rosemary extract, cineol, isopropyl methylphenol, benzethonium chloride, cetylpyridinium chloride, Platycodon Root extract, chlorhexidine hydrochloride, cinnamon extract, thymol, triclosan, hinokitiol, popiyon-iodine, sodium lauroyl sarcosine, phosphorylated oligosaccharide calcium, sodium fluoride, sodium monofluorophosphate, CPP-ACP, hydroxyapatite, calcium, malic acid, dextranase enzyme, calcium glycerophosphate, chlorpheniramine maleate, methyl salicylate, potassium nitrate, Citrus unshiu peel, triamcinolone acetonide, coix seed extract, tranexamic acid, sodium azulene sulfonate, lysozyme chloride, chamomile tincture, licorice extract, diphenhydramine hydrochloride, dipotassium glycyrrhizinate, monoammonium glycyrrhizinate, glycyrrhetinic acid, Shikon extract, prednisolone, Krameria triandra tincture, silica, sodium lauryl sulfate, pyridoxine hydrochloride, sodium chlorite, phytic acid, polyethylene glycol, ethylene glycol, propylene glycol, glycerin, copper chlorophyllin sodium, parsley oil, sunflower oil, green tea flavonoid, rubus extract, laccase, Kiwi powder, Chaenomeles sinensis fruit extract, polyglutamic acid, glucomannan, zinc chloride, gallotannin, tocopherol acetate, probiotics such as lactic acid bacteria and bifidobacteria; other herbs, other vitamins, other oils, functional ingredients contained in other health foods; ingredients contained in drugs and quasi drugs. Additionally, the edible film of the present invention may contain ingredients for the purpose of improving the flavor of the edible films such as sweeteners such as erythritol, maltitol, sorbitol, sucralose, and the like; flavoring, coloring, etc.

The present invention may further employ the following constitutions:

[1] Use of lactoperoxidase, glucose oxidase, a glucose source, and a film former in the manufacture of an edible film for one or more applications selected from the group consisting of intraoral sterilization, sulfur-containing amino acid lyase inhibitor, anti-virus, protection of upper respiratory tract, removal of foreign objects from the upper respiratory tract, prevention of foreign objects from invading into the upper respiratory tract, moisturizing of the upper respiratory tract, suppressing mucosal drying of the upper respiratory tract, reducing mucosal irritation in the upper respiratory tract, improvement of oral flora and improvement of saliva (for example, the amount of saliva, viscosity and smooth sensation of the saliva).

[2] Use of lactoperoxidase, glucose oxidase, a glucose source, and a film former for one or more applications selected from the group consisting of intraoral sterilization, sulfur-containing amino acid lyase inhibitor, anti-virus, protection of upper respiratory tract, removal of foreign objects from the upper respiratory tract, prevention of foreign objects from invading into the upper respiratory tract, moisturizing of the upper respiratory tract, suppressing mucosal drying of the upper respiratory tract, reducing mucosal irritation in the upper respiratory tract, improvement of oral flora and improvement of saliva (for example, the amount of saliva, viscosity and smooth sensation of the saliva).

[3] Lactoperoxidase, glucose oxidase, a glucose source, and a film former used in one or more applications selected from the group consisting of intraoral sterilization, sulfur-containing amino acid lyase inhibitor, anti-virus, protection of upper respiratory tract, removal of foreign objects from the upper respiratory tract, prevention of foreign objects from invading into the upper respiratory tract, moisturizing of the upper respiratory tract, suppressing mucosal drying of the upper respiratory tract, reducing mucosal irritation in the upper respiratory tract, improvement of oral flora and saliva (for example, the amount of saliva, viscosity and smooth sensation of the saliva).

[4] Lactoperoxidase, glucose oxidase, a glucose source, and a film former, which are used for prevention or treatment of one or more diseases or symptoms selected from the group consisting of periodontal disease, halitosis, aspiration pneumonia, cold symptoms, chill, cold, and hay fever.

### EXAMPLES

The present invention will be described in greater detail below using working examples, but the present invention is not limited to these examples.

### Working Example 1

An edible film was produced based on the composition listed in Table 1. Specifically, a 20-fold amount of purified water was added to pullulan used as a film former, and suspended by stirring to prepare a 5% pullulan solution. 10 g of this preparation was added with 14 mg of lactoperoxidase, 14 mg of glucose oxidase, 70 mg of glucose, and as other components, 3.5 mg of menthol and 3.5 mg of flavor to prepare a film stock solution. 10 g of the film stock solution was applied onto a plastic petri dish of 9.5 cm in diameter and dried under a clean environment for 1 day at 30° C. to obtain a monolayered edible film with a film thickness of 50 μm.

TABLE 1
Composition of an edible film (monolayer)

| Raw material name | Composition (mass %) |
|---|---|
| Water | 10 |
| Pullulan | 75 |
| Lactoperoxidase | 2 |
| Glucose oxidase | 2 |
| Glucose | 10 |
| Menthol | 0.5 |
| Flavor | 0.5 |

### Working Example 2

Based on the composition listed in Table 2, an edible film (two layers: layer A and layer B) was manufactured. Specifically, a 20-fold amount of purified water was added to the pullulan used as a film former, and suspended by stirring to prepare a 5% pullulan solution. 10 g of this preparation was added with 70 mg of glucose as the active ingredient to be used as a film raw material of layer A (Raw Material Composition A). Similarly, 10 g of the prepared solution described above was added with 14 mg of lactoperoxidase, 14 mg of glucose oxidase, and as another components, 3.5 mg of menthol and 3.5 mg of flavor to prepare a film raw material of layer B (Raw Material Composition B).

5 g of the film raw material of layer A was applied to a plastic petri dish of 9.5 cm in a diameter and dried under a clean environment for 1 day at 30° C. to obtain a film of layer A. Subsequently, 5 g of film stock solution of layer B was applied onto the layer A film, and under a clean environment, it was dried at 30° C. for 1 day to obtain an edible film having two layers (50 μm in film thickness).

TABLE 2
Composition of edible film (two layers)

| | Composition | |
|---|---|---|
| Raw material name | (mass %) | Layer |
| Water | 10 | A and B |
| Pullulan | 75 | A and B |
| Lactoperoxidase | 2 | B |
| Glucose oxidase | 2 | B |
| Glucose | 10 | A |
| Menthol | 0.5 | B |
| Flavor | 0.5 | B |

### Working Example 3

A film of layer A containing glucose obtained in Working Example 2 was used to produce an edible film in the form of capsules encapsulating lactoperoxidase powder and glucose oxidase powder. Specifically, a 20-fold amount of purified water was added to the pullulan used as a film former, and suspended by stirring to prepare a 5% pullulan solution. 10 g of this preparation was added with 70 mg of glucose as the active ingredient to prepare as a film raw material of layer A (Raw Material Composition A). 5 g of the film raw material of layer A was applied in a plastic petri dish of 9.5 cm in diameter and dried under a clean environment for 1 day at 30° C. to obtain a layer A film. In this layer A film, a powder composition comprising 100 mg of lactoperoxidase and 100 mg of glucose oxidase (Raw Material Composition B) was encapsulated in a capsule form and bonded to films to obtain a capsule-shaped, edible film.

### Working Example 4

Based on the composition listed in Table 3, an edible film (two layers: layer A and layer B) containing lactoferrin was manufactured. Specifically, a 20-fold amount of purified water was added to the pullulan used as a film former, and suspended by stirring to prepare a 5% pullulan solution. 10 g of this preparation was added with 70 mg of glucose as the active ingredient to prepare a film raw material of layer A (Raw Material Composition A). Similarly, 10 g of the preparation was added with 35 mg of lactoferrin, 14 mg of lactoperoxidase, 14 mg of glucose oxidase, and as other components, 3.5 mg of menthol and 3.5 mg of flavor to prepare a film raw material of layer B (Raw Material Composition B). 5 g of the film raw material of layer A was applied to a plastic petri dish of 9.5 cm in a diameter and dried under a clean environment for 1 day at 30° C. to obtain a layer A film. Subsequently, 5 g of film stock solution of layer B was applied onto layer A film, and under a clean environment, it was dried at 30° C. for 1 day to obtain an edible film having two layers (50 μm in film thickness).

TABLE 3
Composition of edible film (two layers)

| | Composition | |
|---|---|---|
| Raw material name | (mass %) | Layer |
| Water | 10 | A and B |
| Pullulan | 70 | A and B |
| Lactoperoxidase | 2 | B |
| Glucose oxidase | 2 | B |
| Glucose | 10 | A |
| Lactoferrin | 5 | B |
| Menthol | 0.5 | B |
| Flavor | 0.5 | B |

### Comparative Example

Tablet confectionaries were made based on the composition listed in Table 4. Each powder of 100 g of erythritol, 350 g of starch syrup of reduced malt sugar, 150 g of sorbitol, 100 g of corn starch, 20 g of citric acid, 5 g of trisodium citrate, 85 g of xylitol, 40 g of sucrose fatty acid ester, 20 g of lactoperoxidase, 20 g of glucose oxidase, 100 g of glucose, 5 g of menthol, and 5 g of flavoring were uniformly mixed together, and a tableting machine was used to perform continuous tableting of the mixed powder described above, 200 mg per tablet at a tableting speed of 12 tablets/minutes and at a pressure of 9.8 Kpa to produce 5000 tablet confectioneries (about 1000 g).

TABLE 4
Combination for tablet confectionary

| | Composition |
|---|---|
| Raw material name | (mass %) |
| Erythritol | 10 |
| Starch syrup of reduced malt sugar | 35 |
| Sorbitol | 15 |
| Corn starch | 10 |
| Citric acid | 2 |
| Trisodium citrate | 0.5 |
| Xylitol | 8.5 |
| Sucrose fatty acid ester | 4 |
| Lactoperoxidase | 2 |
| Glucose oxidase | 2 |
| Glucose | 10 |
| Menthol | 0.5 |
| Flavor | 0.5 |

### Test Example 1

The edible films manufactured in Working Example 1 and Working Example 2 were cut into 2×3 cm (200 mg in weight) each, each of which was sealed in an aluminum pouch bag and stored at 37° C. The film was removed after a specified storage period, and the lactoperoxidase activity and glucose oxidase activity were measured by the following procedure, and the results are shown in Table 5.

Measurement of Lactoperoxidase Activity

The measurement was performed in reference to the method by Putter et al. (1983, Methods of Enzymatic Analysis (Bergmeyer, H. U. ed) Volume III, Third Edition, pp. 286-293, Verlag Chemie, Deerfield Beach, Fla.). 200 mg of an edible film was dissolved in 30 ml of 200 mM dipotassium phosphate solution to prepare a sample. A mixed solution comprising 2.2 ml of 100 mM potassium phosphate buffer solution (pH 5. 5), 0.70 ml of 100 mM ABTS solution, and 0.1 ml of 0.025% hydrogen peroxide solution was poured into the cells, after which 0.05 ml of sample was added to measure changes in absorbance at 25° C. and 412 nm. In a reaction solution, 23 mM of the ABTS was included. In pH 5.5, at 25° C., the amount of enzyme required to oxidize 1 μM of ABTS per minute was defined as 1 unit.

Measurement of Glucose Oxidase Activity

The measurement was performed in reference to the method by Bergmeyer et al. (1974, Methods of Enzymatic Analysis (H.U. ed) Volume I, Second Edition, pp. 457-458, Academic Press Inc., New York, N.Y.). 200 mg of the edible film was dissolved in 15 ml of 50 mM sodium acetate buffer solution (pH 5.1) to prepare a sample. 3.0 ml of mixed solution comprising 2.4 ml of 0.21 mM o-dianisidine solution, 0.5 ml of 10% glucose solution, 0.1 ml of 60 units/ml horseradish peroxidase was poured into the cells, a 0.1 ml sample was added to measure the changes in absorbance at 35° C. and 500 nm. The reaction solution contains glucose at a concentration of 90 mM. In pH 5.1, at 35° C., the amount of enzyme required to oxidize 1 μM glucose to gluconolactone and hydrogen peroxide per minute was defined as 1 unit.

TABLE 5

| | Working Example 1 | Working Example 2 |
|---|---|---|
| | (A monolayer film) | (A two-layer film) |
| Lactoperoxidase | 10 | 55 |
| activity (units/g) | | |
| Glucose Oxidase | 25 | 45 |
| activity (units/g) | | |

As shown from the results in Table 5, it was found that a two-layer film sample (Working Example 2) containing glucose oxidase and a glucose source in separate layers is slower in a decrease of lactoperoxidase activity and glucose oxidase activity compared to those of a monolayered film sample (Working Example 1) in which glucose oxidase and a glucose source were blended into the same layer.

### Test Example 2

After 8 monitors ingested an edible film containing lactoferrin, lactoperoxidase, glucose oxidase, and a glucose source prepared in Working Example 4 and the tablet confectionary prepared in the comparative example, the film was dissolved in the oral cavity of all monitors, but the dissolution rate was slow, and the film remained in the oral cavity for at least 10 minutes. On the other hand, the remaining time of the tablet confectionary in the mouth was less than or equal to 3 minutes.

### Test Example 3

In this test example, we tested the effect of the film-forming materials used in the edible film of the present invention on the activity of glucose oxidase and the activity of lactoperoxidase.

Here, glucose oxidase generates hydrogen peroxide from glucose and oxygen. Lactoperoxidase also catalyzes the generation of hypothiocyanic acid and water from hydrogen peroxide and thiocyanate. Based on this fact, this study confirmed how the activity of lactoperoxidase and the activity of glucose oxidase differed by the materials of the film former by measuring the amount of hypothiocyanic acid generated.

The quantification of hypothiocyanic acid was carried out in reference to the method by Tenovuo et al. (1985, Antibacterial effect of lactoperoxidase and myeloperoxidase against Bacillus cereus Antimicrob. Agents Chemother. 27 96-101). As an indicator for this quantification, the one prepared as follows was used: 50 ml of 50 mM phosphate buffer solution (pH 7.2) was added with 0.1 mM of diethylenetriaminepentaacetic acid, and then 20 mg of 5,5′-dithiobis (2-nitrobenzoic acid) and 1 ml of 0.2% mercaptoethanol were added.

Nine types of a film former were used: pullulan, hydroxypropyl cellulose (HPC), hydroxypropyl methylcellulose (HPMC), sodium alginate, pectin, processed starch, carrageenan, and porphyran. Each film former was added with a 200-fold amount of 0.6 mM of thiocyanic acid solution, stirred and suspended to prepare a 0.5% film-forming solution. In 10 g of this prepared solution, 30 mg of glucose, 20 mg of lactoferrin, 2 mg of lactoperoxidase, and 25 mg of glucose oxidase were dissolved and incubated at 37° C. for 10 minutes. Furthermore, 100 μl of the dissolution liquid was diluted with cooled 1900 μl of 50 mM phosphate buffer solution (pH 7.2), the aforementioned indicator was added, and the production amount of hypothiocyanic acid was measured from the changes in absorbance at 409 nm.

As a result, when the amount of hypothiocyanic acid in the aqueous solution in which no film former was added was set to 100%, the amount of hypothiocyanic acid generated in each case is as shown in Table 6 below.

TABLE 6

| | Generation amount of |
|---|---|
| Film former | hypothiocyanic acid (%) |
| None | 100 |
| Pullulan | 79.7 |
| HPC | 87.9 |
| HPMC | 54.0 |
| Sodium alginate | 23.1 |
| Pectin | 23.8 |
| Processed starch | 100 |
| Carrageenan | 43.9 |
| Porphyran | 90.9 |

It was found from the results shown in Table 6 that the activity of glucose oxidase and the activity of lactoperoxidase were demonstrated in any film former; however, as the film former, when pullulan, hydroxypropyl cellulose (HPC), hydroxypropyl methylcellulose (HPMC), processed starch, and porphyran were used, the activity of glucose oxidase and the activity of lactoperoxidase were larger than those of other materials.

It is apparent from the results above that an edible film comprising lactoperoxidase, glucose oxidase, a glucose source, and a film former, as well as an edible film comprising lactoferrin, lactoperoxidase, glucose oxidase, a glucose source, and a film former are convenient to carry and can be kept for a long time in the oral cavity. It has also been revealed that in an edible film comprising lactoperoxidase, glucose oxidase, a glucose source, and a film former, when the glucose source and glucose oxidase are not included in the same layer, a decrease in lactoperoxidase activity and glucose oxidase activity is more suppressed as compared with when the edible film is monolayered.

### INDUSTRIAL AVAILABILITY

An edible film comprising lactoperoxidase, glucose oxidase, a glucose source, and a film former, as well as an edible film comprising lactoferrin, lactoperoxidase, glucose oxidase, a glucose source, and a film former can be applied to food, pharmaceutical products, and the like.

1. An edible film comprising lactoperoxidase, glucose oxidase, a glucose source, and a film former.

2. The edible film according to claim 1, wherein the edible film is monolayered.

3. The edible film according to claim 1, wherein the edible film is multilayered and does not include the glucose source and the glucose oxidase in the same layer.

4. The edible film according to claim 1, wherein the edible film has a lactoperoxidase content of 0.02 to 20 wt %, a glucose oxidase content of 0.02 to 20 wt %, and a glucose source content of 0.02 to 40 wt %.

5. The edible film according to claim 1, further comprising lactoferrin.

6. The edible film according to claim 5, wherein the edible film has a lactoferrin content of 0.02 to 40 wt %.

7. The edible film according to claim 1, wherein the film former is selected from the group consisting of pullulan, hydroxypropyl cellulose (HPC), hydroxypropyl methylcellulose (HPMC), processed starch, porphyran, and combinations thereof.

8. A method for manufacturing an edible film, the method comprising:
a step of forming into a film a mixture comprising lactoperoxidase, glucose oxidase, a glucose source, and a film former.

9. A method for manufacturing an edible film, the method comprising:
a step of forming into a film either a first mixture or a second mixture, the first mixture comprising glucose oxidase without any glucose source, the second mixture comprising a glucose source without glucose oxidase; and
a step of laminating, onto either the first mixture formed into the film, the second mixture; or the second mixture formed into the film, the first mixture, wherein
either or both of the first mixture and the second mixture include lactoperoxidase, and
either or both of the first mixture and the second mixture include a film former.

10. A method for manufacturing an edible film, the method comprising:
a step of forming into a film a first mixture comprising glucose oxidase without any glucose source;
a step of forming into a film a second mixture comprising a glucose source without glucose oxidase; and
a step of laminating the first mixture formed into the film and the second mixture formed into the film, wherein
the order in which the step of forming the first mixture into the film and the step of forming the second mixture into the film are performed is not limited,
either or both of the first mixture and the second mixture include lactoperoxidase, and
either or both of the first mixture and the second mixture include a film former.
